(19) United States
(12) Patent Application Publication
(10) Pub. No.: US 2007/0237815 A1
(43) Pub. Date: Oct. 11, 2007

(54) DOSAGE FORMS AND METHODS COMPRISING AMLODIPINE AND CHLORTHALIDONE

(76) Inventors: Lawrence Solomon, Boca Raton, FL (US); Allan S. Kaplan, Boca Raton, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 11/696,019

(22) Filed: Apr. 3, 2007

Related U.S. Application Data
(60) Provisional application No. 60/790,002, filed on Apr. 6, 2006.

(51) Int. Cl. A61K 9/44; A61K 9/48

(52) U.S. Cl. 424/451; 424/467

(57) ABSTRACT

The subject invention provides compositions and methods of use of a single dosage form which includes therapeutic or subtherapeutic doses of a dihydropyridine calcium channel blocker (CCB), such as amlodipine, and a diuretic, preferably a thiazide-type diuretic, such as chlorthalidone.

### CROSS REFERENCE TO RELATED APPLICATIONS

This is a continuation-in-part of U.S. Provisional Patent Application Ser. No. 60/790,002, filed Apr. 6, 2006.

### FIELD OF THE INVENTION

This invention is directed to pharmaceutical compositions comprising a calcium channel blocker and a diuretic, preferably amlodipine and chlorthalidone, and to methods of treating hypertension using both drugs in combination.

### BACKGROUND OF THE INVENTION

Amlodipine is a dihydropyridine calcium antagonist (calcium channel blocker or CCB) which has anti-hypertensive and anti-anginal activity. Chlorthalidone, a long-acting diuretic, has been known for decades as an effective treatment to lower elevated systemic arterial blood pressure (“hypertension”, as distinguished from pulmonary arterial hypertension, portal venous hypertension, or the like).

Suggestions of combining amlodipine with a diuretic, such as chlorthalidone, to treat hypertension have been controversial and a source of dispute among experts. For example, the seventh report of the U.S. Joint National Committee on the Prevention, Detection, Evaluation and Treatment of High Blood Pressure (“JNC VII”) recommends the concurrent use of a CCB and a “thiazide-type⇄ diuretic as a possible co-administration regimen; however, the British Hypertension Society recommends that a CCB and a diuretic represents an illogical combination for hypertension treatment.

JNC VII does not specify any particular CCB or thiazide-type diuretic to be co-administered, nor does it call for any co-formulation of the compounds. Other studies have evaluated patients receiving both amlodipine and hydrochlorothiazide (“HCTZ”), and two additional studies have evaluated a different CCB (nifedipine) plus chlorthalidone—all with equivocal results.

The CCB, amlodipine, is currently available in commercial drug products, typically in the form of its pharmaceutical salt. For example, amlodipine besylate, is the active pharmaceutical ingredient in the drug product marketed in the United States under the trade name Norvasc®, which is sold in 2.5 mg, 5.0 mg, and 10.0 mg base-equivalent strengths. A typical dose range for Norvasc is 5-10 mg daily for hypertension and angina, except in the frail, small-statured, or elderly; or in pediatric use. A combination product containing amlodipine besylate and an angiotensin converting enzyme (ACE) inhibitor, benazepril, is available commercially in the U.S., marketed under the trade name of Lotrel®.

The chemical compound, chlorthalidone, was patented in U.S. Pat. No. 3,055,904, and disclosed a total daily oral dose of 50 mg to 200 mg per day, dosed from one to three times a day or, alternatively, 100 mg dosed every second day. In U.S. Pat. No. 5,948,799, chlorthalidone is described as being dosed at 6.25-200 mg daily, and is dosed in a preferred range of 12.5 to 100 mg daily for reducing morbidity and mortality in heart failure patients. The '799 patent also discloses this use of chlorthalidone in combination with amlodipine and/or digoxin for heart failure. There is no mention in this '799 patent of the use of chlorthalidone and amlodipine for the treatment of hypertension.

Chlorthalidone has been extensively tested in the United States. Some of the National Institutes of Health (NIH) major studies that have evaluated chlorthalidone include:
Multiple Risk Factor Intervention Trial (MRFIT),
Treatment of Mild Hypertension Study (TOMHS),
Hypertension Detection and Follow-Up Program (HDFP),
Systolic Hypertension in the Elderly Program (SHEP), and
Antihypertensive and Lipid-Lowering Treatment to Prevent Heart Attack Trial (ALLHAT).

The above-referenced NIH studies used doses that are higher than the lower doses described for the subject invention. For example, the SHEP and ALLHAT studies used chlorthalidone at starting doses of 12.5 mg per day, raising said dose to 25 mg in the majority of cases that generally did not achieve goal blood pressure (BP) at 12.5 mg chlorthalidone daily as monotherapy. Despite these studies, and perhaps because the lowest marketed dose of chlorthalidone is an unscored 25 mg tablet in the U.S., physician prescriptions for this drug have been quite limited.

Heretofore, no co-formulated immediate-release or once-a-day product containing both a dihydropyridine, e.g., amlodipine, and the thiazide-type diuretic, chlorthalidone, has been described or successfully produced. A sustained-release, BID composition containing diltiazem plus HCTZ has been reported, but no product was successfully launched. Diltiazem, though a CCB, is not a dihydropyridine and has different clinical effects from amlodipine. Accordingly, the usefulness of combining amlodipine with chlorthalidone in a single dosage form, and at particular doses as described herein, has not been specifically disclosed or suggested by the prior art.

### SUMMARY OF THE INVENTION

In a preferred embodiment, the subject invention provides compositions and methods of use of a single dosage form consisting essentially of therapeutic or subtherapeutic doses of a dihydropyridine calcium channel blocker (CCB)), such as amlodipine, and a diuretic, preferably a thiazide-type diuretic, such as chlorthalidone. Alternatively, the method of the subject invention can comprise providing both active ingredients in a combined packaging. The compositions used in accordance with the subject invention may be formulated for oral, rectal, transdermal, intravenous, or other routes of administration according to techniques that are well known to those who are skilled in the pharmaceutical arts. Dosage ranges, or strengths, of the compositions of the invention are directed toward the administration aid treatment of adult patients with hypertension. Preferred daily doses include amlodipine doses of from 1-20 mg and chlorthalidone doses of from 2-50 mg; more preferred daily doses are: amlodipine 2-15 mg and chlorthalidone 3-25 mg; and most preferred daily doses are amlodipine 2.5-10 mg and chlorthalidone 3.25-25 mg.

With regard to the treatment of hypertension, the administration of both amlodipine and chlorthalidone, according to the subject invention can unexpectedly provide an advantageous peak-to-trough ratio as compared to the peak-to-trough ratio obtained by the administration of either amlodipine or chlorthalidone alone.

In hypertensive patients, the smoothness index (“SI”) may also be improved by the administration of amlodipine and chlorthalidone according to the invention, as compared to the administration of either amlodipine or chlorthalidone alone. “Improved smoothness index” is a term that is well known in the art and used to describe the homogeneity of the blood pressure readings when blood pressure reduction is induced by antihypertensive treatment over a 24 hours period. The (SI) may for example be calculated by (1) calculating the average of blood pressure values for each hour of the 24 hour monitoring period, both before and during treatment; and all hourly changes in blood pressure induced by treatment, (2) computing the average and standard deviation of the hourly blood pressure changes, and (3) obtaining the SI.

Amlodipine and chlorthalidone administered in accordance with the subject invention may also advantageously ameliorate peripheral edema—the most common side effect of amlodipine—without further side effects other than hypokalemia that is known to be generally mild. Peripheral edema is believed to arise from the peripheral vasodilatory action of amlodipine, and is not associated with salt or water retention. Lotrel has been shown to ameliorate the edema associated with amlodipine, presumably due to the presence of benazapril, the ACE inhibitor component of Lotrel. Benazepril, however, is not classified as a diuretic. A recent article by an expert in hypertension offers the opinion that in certain instances it is “ill-advised to routinely diurese patients with CCB-related peripheral edema for the sole purpose of correcting the edema state.” Sica, D A, (2003) “Calcium Channel Blocker-related Peripheral Edema: Can it be Resolved?” J. Clin. Hypertens 5(4):291-294, 297. Thus, diuretics may be contraindicated for use with CCBs such as amlodipine.

In the most preferred embodiments of the subject invention, the invention is a capsule or a tablet for oral ingestion. Formulations and manufacturing techniques for tablets and capsules are well known in the art, and are described, for example, in Remington's Pharmaceutical Sciences 20th Ed., Mack Publishing Co., Easton, Pa. (2000), which is incorporated herein by reference. For purposes of the subject invention, the term “concentration” refers to a weight to weight basis, unless specified otherwise. Preferred tablet embodiments, which are depicted in the drawings attached hereto, without limitation, include any of the following tablet structures:

A: Layered Tablet Comprising Two Active Segments and an Interposed Inactive Segment, Comprising:
1. a first segment comprising a therapeutically effective amount of amlodipine and lacking a therapeutically effective quantity of chlorthalidone;
2. a second segment interposed between the first segment and a third segment, the second segment comprising a composition substantially lacking pharmacological activity, or a diminished concentration of amlodipine relative to said first segment or a diminished concentration of chlorthalidone relative to a third segment; and
3. a third segment comprising substantially a therapeutically effective amount of chlorthalidone.

B: Layered Tablet Comprising Two Combination Segments and an Interposed Inactive Segment, Comprising:
1. a first segment comprising therapeutically effective quantities of both amlodipine and chlorthalidone;
b 2. a second segment interposed between said first segment and a third segment, the second segment substantially lacking pharmacological activity or containing a diminished concentration of both amlodipine and chlorthalidone compared to said first and third segments; and
3. Said third segment comprising therapeutically effective quantities of both amlodipine and chlorthalidone in the same ratio as in said first segment; and

C. Layered Tablet Comprising Two Active Segments and an Inactive “Support” Segment with:
1. a first and a second segment comprising substantially equal concentrations of both amlodipine and chlorthalidone, each segment adjoining the same face of
2. a third “support” segment that preferably lacks a pharmacologically effective quantity of a drug or at most contains a diminished concentration of both amlodipine and chlorthalidone relative to said first and second segments.

These and other embodiments of the invention, certain of which are described in PCT applications WO 2005/112870 WO 2005/112897, WO 2005/112998, and WO 2005/112900, would be readily recognized by persons of ordinary skill in the art as applicable to the subject invention, and are hereby incorporated by reference in their entirety.

A segment, e.g., a middle segment lacking a pharmacologically effective quantity of an active ingredient, can have a score or a printed mark such as a line that will locate a preferred zone for breaking the tablet apart, wherein the breaking occurs substantially only through that scored or printed segment.

Tablets or capsules where the amlodipine and chlorthalidone are in admixture and that lack a segment that lacks a pharmacologically effective quantity of a drug also comprise preferred embodiments. Other embodiments of the subject invention may include all of the known modes of administering pharmaceuticals, such as transdermal, buccal, rectal, intravenous, intramuscular, and the like.

In general, lower doses of amlodipine are preferred to be co-formulated with lower doses of chlorthalidone, and higher doses of amlodipine tend to be more useful with higher doses of chlorthalidone. More preferred daily doses for adults include, for example, 6 mg of chlorthalidone with 2.5-7.5 mg of amlodipine, 12-25 mg of chlorthalidone with 5-10 mg of amlodipine, and 3-6.25 mg of chlorthalidone with 2-5 mg of amlodipine.

The subject invention also includes novel dosing regimens. Typically, treatment of hypertension using a CCB, such as amlodipine, may comprise prescribing or administering a starting dose for a period of time. This starting dose may then be increased (often doubled) to provide a more effective maintenance dose. Further increases in dose may also be prescribed. For example, a physician may initially prescribe a starting dose of 5 mg amlodipine per day. If that starting dose is determined to be inadequate for reaching a therapeutic or clinical goal, e.g., it is insufficient reduction of blood pressure or amelioration of a hypertensive condition, the daily dose of amlodipine may then be increased to 10 mg per day.

By contrast, rather than increasing the dose of the CCB (amlodipine), an embodiment of the subject invention comprises prescribing or administering a starting dose of a CCB, such as amlodipine, for a period of time, followed by, if needed, a prescribed administration of that starting dose of the CCB in combination with a starting dose of a thiazide-like diuretic. For example, the subject invention can comprise prescribing or administering a starting dose of 5 chlorthalidone, rather than doubling the dose of amlodipine to 10 mg per day.

Alternatively, the starting dose of the diuretic may be followed by a combination of diuretic plus CCB rather than, as typically prescribed, increasing or doubling the dose of the diuretic. In this embodiment, for example, chlorthalidone is prescribed or administered to the patient at a starting dose of 12.5 mg per day for 7-14 days, followed by, if needed, a prescribed administration of 12.5 mg per day of chlorthalidone in combination with 5 mg per day of amlodipine.

It would be recognized that the above dosing regimens of the subject invention can be carried out using any effective, therapeutic or subtherapeutic dose of the CCB or diuretic. Preferably, however, the starting doses of the active ingredient, used as a monotherapy or in the combinations, are doses that are lower than typically prescribed or used as maintenance doses. These regimens can be useful even where the therapeutic effect of one or both of the starting doses is statistically insignificant and are advantageous in that the combination of the active ingredients provide a statistically significant effect. Another advantage of the compositions and method of the subject invention is that these lower starting doses can minimize side effects that have been observed or may be expected from the use of higher doses of one or more of the active ingredients.

In addition, the dosing regimens of the subject invention as described herein can advantageously be carried out using a single dosage form that contains both actives in combination, and that are breakable in a manner which substantially separates one active ingredient from the other active ingredient. For example, the subject dosing regimen can be facilitated using a dosage form comprising a first segment comprising a starting dose of amlodipine, a second segment consisting essentially of inactive ingredient wherein the second segment is disposed between the first segment and a third segment comprising a starting dose of chlorthalidone. In that way, either of the two actives can be administered alone, as a starting dose, by breaking through the substantially inactive segment of the tri-segmented tablet. If this starting dose of the single agent is insufficient, the whole tablet comprising both actives can then be prescribed and administered.

### BRIEF DESCRIPTION OF THE DRAWINGS

FIG. 1 is a partially exploded perspective view of a dosage form in which a first segment comprises a therapeutically effective quantity of amlodipine, a third segment comprises a therapeutically effective quantity of chlorthalidone, and a second segment comprises a lesser quantity of both drugs is interposed between said first and third segments.

FIG. 2 is a partially exploded perspective view of a dosage form in which a first and third segment comprise compositionally similar ratios of amlodipine and chlorthalidone and a second segment comprises a lesser quantity of both of said drugs exists between said first and third segments.

FIG. 3 is a cross-sectional front view of a tablet having a first and second segment that are compositionally substantially identical and that comprise both amlodipine and chlorthalidone and that adjoin a third segment that contains a lesser quantity of both of said drugs.

### DETAILED DESCRIPTION OF THE INVENTION

The invention provides numerous unexpected benefits. One such benefit relates to the pharmacokinetics of each drug. Both amlodipine and chlorthalidone have approximately 40-48 hour half-lives in normal adult patients; the half-lives of both drugs increase to approximately 72 hours in the elderly. Under steady state conditions, the combination will exhibit an enhanced pharmacodynamic activity. The composition comprising a combination of these active ingredients thus may have unexpected effects on duration and pharmacokinetic effect, such as improved peak-to-trough ratio and the Smoothness Index (SI). For example, if a 12.5 mg dose of chlorthalidone reduces systolic blood pressure by 10 mm Hg, adding a dose of amlodipine that typically also reduces systolic blood pressure 10 mm Hg will produce a greater reduction in systolic blood pressure than adding another 12.5 mg dose of chlorthalidone (i.e., doubling the dose of chlorthalidone to 25 mg). Peak-to-trough ratio and SI resulting from the combination of amlodipine and chlorthalidone will also improve as compared to doubling the dose of chlorthalidone.

Conversely, if the starting dose of amlodipine is, for example 2.5 mg, and lowers blood pressure 8 mm Hg, adding a 6.25 mg dose of chlorthalidone can provide a superior blood pressure-lowering effect as compared to adding a second 2.5 mg dose of amlodipine. Peak-to-trough ratio and SI can also be improved by use of the combination as compared to use of a first and second dose of amlodipine.

Another unexpected advantage of the subject invention is the ability of chlorthalidone to favorably ameliorate peripheral edema caused by amlodipine. It has been observed that the rate of edema triples or quadruples as the dose of amlodipine doubles from 5 to 10 mg daily. The additive, and at times synergistic, effects of the co-formulation of amlodipine and chlorthalidone can thus provide compositions and methods which result in fewer side effects than would be caused by giving a higher dose of either drug alone.

A hypertensive patient treated with a low daily dose of each drug in a co-formulation, such as chlorthalidone 12.5 mg and amlodipine 2.5 mg, dosed once daily can exhibit an anti-hypertensive effect equal to that of a daily dose of chlorthalidone 25 mg, taken alone or a daily dose of amlodipine 10 mg taken alone.

It is believed that ultra-low doses of each drug, such as 2 mg of amlodipine and 3-6 mg of chlorthalidone, which may be relatively ineffective when given separately, are effective in combination, which can support a synergistic benefit.

To manufacture the invention, different tablets have been disclosed above. In a standard tablet or capsule lacking an inactive segment, amlodipine or a suitable salt thereof, of which besylate is the most preferred salt, is provided with chlorthalidone and suitable excipients and either is placed in a capsule or utilized in a standard fashion, such as via direct compression or utilizing a wet granulation, into a tablet. It would be understood that the active ingredients, amlodipine and chlorthalidone, can be used as a free base, or as any suitable derivative, hydrate, metabolite, polymorphic form, or salt thereof.

In a preferred embodiment of the novel tablet, amlodipine and chlorthalidone are formulated separately with suitable excipients. A suitable inactive segment is formulated, and the tablet is prepared on a tri-layer or five-layer press, such as a commercially available multi-layer tablet press manufactured by Korsch AG of Germany. Alternatively, amlodipine and chlorthalidone are co-formulated with suitable excipients and compressed in an appropriate layer press with at least one other layer derived from an inactive composition, e.g., granulation. In the standard tablets and capsules well known in the art, a suitable co-formulation is prepared and either tabletted or encapsulated, and no separately formulated inactive granulation is utilized.

The invention also includes the method of treating hypertension by adding to an amlodipine dosage regimen an amount of chlorthalidone that is sufficient to then lower the blood pressure to below a desired upper limit, such as below 140 mm Hg systolic and below 90 mm Hg diastolic. Also included is the method of treating hypertension by adding to a chlorthalidone dosage regimen an amount of amlodipine that is sufficient to then lower the blood pressure to below the 140/90 mm Hg range described immediately above.

One of the specific embodiments of the invention is the method of treatment of hypertension with an initial dose of amlodipine that is a sub-maximal dose, such as 5 mg daily, in which either blood pressure response is inadequate or edema occurs. The method of the subject invention is to begin treatment with the novel composition of amlodipine plus chlorthalidone (or pack thereof). It has however been taught to begin treatment with a diuretic and follow it with a CCB if response to the diuretic is inadequate. In the case of chlorthalidone at doses of less than 25 mg, it has not been taught to begin with such a low dose and then add a CCB if BP response is inadequate, and it further has not been taught to co-administer, such as with a co-formulation or with the aid of a pack, a sub-25 mg dose of chlorthalidone plus a CCB such as amlodipine.

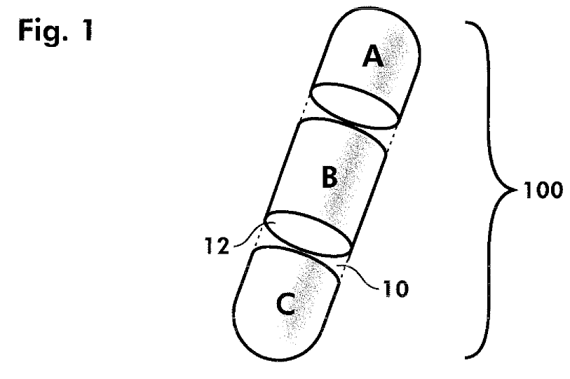

FIG. 1 depicts a preferred tablet 100 of the invention. As shown in the exploded external view, said tablet 100 has three distinct segments which are adjoined in the tablet as manufactured. Segment A comprises a therapeutic quantity of amlodipine, segment B is pharmacologically inactive and segment C comprises a therapeutic quantity of chlorthalidone. Segment A lacks a pharmacologically effective quantity of chlorthalidone but may not be completely devoid of it, due to slight intermixing of granulations. Preferably, both the amlodipine and chlorthalidone compositions are formulated as wet granulations to minimize said intermixing of granulations. Space 10 shows the alignment of segment B and segment C which adjoin one another. Interface 12 depicts somewhat schematically the region at which the inactive granulation forming segment B and the granulation comprising chlorthalidone that forms segment C intermix during the powder filling and compression process.

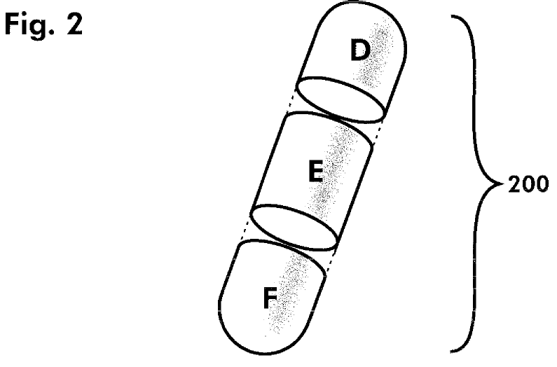

FIG. 2 depicts another preferred tablet 200 of the invention. As shown in the exploded external view, said tablet 200 has three distinct segments which are adjoined in the tablet as manufactured. Segment D comprises a therapeutic quantity of amlodipine and chlorthalidone, segment E is pharmacologically inactive and segment F comprises a therapeutic quantity of amlodipine and chlorthalidone, preferably in substantially the same quantity and at a substantially similar ratio as the mixture in segment D.

The amounts of amlodipine and chlorthalidone that may be used in tablets of the above structure can vary depending on the dosage regimen which is adopted for a particular patient. In general the dose of amlodipine in this dosage form may be within 1 to 20 mg and a preferable dose range of chlorthalidone is 3 to 6 mg but a preferred dose of chlorthalidone may be 3 to 50 mg depending on the particular patient. The elongated segment 4 is long enough to be easily broken by hand and may optionally contain a score or printed indicia to facilitate optional tablet breaking through segment 4 without impinging upon segments 2 or 6.

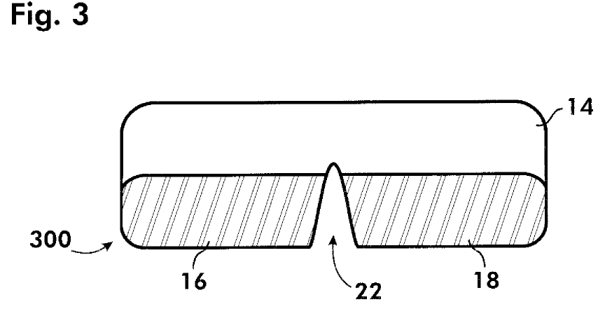

FIG. 3 shows a tablet structure 300 having an upper segment 14 that is substantially inactive and having two lower segments 16 and 18 attached to upper segment 14 at face 20. Deep score 22 is between segments 16 and 18 and as shown, the score extends into upper segment 14 and allows for breakage to take place only in upper segment 14 thus allowing exact division of the active material in the tablet. The score may be formed and placed by selection of an appropriate embossed tablet die or by a post tabletting filing or grinding procedure.

The invention also includes treating hypertension by the administration of amlodipine, chlorthalidone, and one or more of the members of the group consisting of another anti-hypertensive agent, a potassium-retaining diuretic, a cholesterol lowering agent, or a vitamin, additional anti-hypertensive drug such as, without limitation, an angiotensin converting enzyme (ACE) inhibitor (e.g., enalapril, lisinopril, benazepril, ramipril, captopril), an angiotensin receptor blocker (ARB) (e.g., losartan, valsartan, candesartan, eprosartan), or an alpha blocker (e.g., prazosin, doxazosin, terazosin); a calcium antagonist (calcium channel blocker, CCB) (e.g., nifedipine, nicardipine, nisoldipine, amlodipine (racemic), S-amlodipine, lacidipine, lercanidipine, manidipine, azelnidipine); an aldosterone antagonist (e.g., spironolactone, potassium canrenoate, eplerenone); a beta blocker (e.g., carvedilol, nebivolol, atenolol, metoprolol, betaxolol, bisoprolol) at appropriate doses such as the doses that are disclosed in the Physicians' Desk Reference (2005) which is incorporated by reference. The potassium sparing diuretics include without limitation triamterene and amiloride which may be administered according the Physicians Desk Reference (2005). Cholesterol lowering agents include without limitations statins such as lovastatin, simvastatin, atorvastatin, rosuvastatin and pravastatin; ezetimide; and fibrates such as fenofibrate and gemfibrozil. Vitamins include water soluble and oil soluble vitamins such as the B-complex vitamins, folic acid, vitamin C, D, E, K and the like in doses generally acceptable for the prevention or treatment of vitamin deficiencies.

### EXAMPLES

### Example 1

### Active Blend and Inactive Blend Compositions

A. Formulation of Chlorthalidone Active Blend
The following ingredients were used at the specified weight percentages to formulate a chlorthalidone active blend composition:

| Ingredient | Wt. % |
|---|---|
| chlorthalidone | 6.67 |
| dibasic calcium phosphate, anhydrous | 15.31 |
| microcrystalline cellulose PH 102 | 67.06 |
| microcrystalline cellulose PH 105 | 6.67 |
| sodium starch glycolate | 4.08 |
| Red or Blue Lake | 0.01 |
| magnesium stearate | 0.2 |
| Total | 100 |

Step 1. Mixing
a. Chlorthalidone and an equal mass of microcrystalline cellulose (MCC) PH 105 are added into a high shear mixer and mixed for 3 minutes.
b. The mixture from step a, above, is placed in a suitably sized “V” blender. MCC PH 102, sodium starch glycolate and Red or Blue Lake are added to the mixture from step a, and mixed for 15 minutes.
c. Half of the magnesium stearate is added to the mixture from step b, above, and blended for 3 minutes.

Step 2. Roller Compaction
d. The blended mixture from step c is dry granulated on a suitable roller compactor, at a compression force between 8 to 12 kN/cm and at a roller speed of 3 to 6 rpm.
e. The roller-compacted material from step d is milled to a particle size suitable for tablet compression.

Step 3. Mixing of Final Active Blend
f. The milled material from step e is placed in a suitably sized “V” blender. The remaining magnesium stearate is added to the blender and the material is mixed for 3 minutes to obtain the final active blend.

B. Formulation of Inactive Blend
The following ingredients were used at the specified weight percentages to formulate an inactive blend composition:

| | Wt. % | Wt. % |
|---|---|---|
| Ingredient | (granulation) | (Final Blend) |
| dibasic calcium phosphate, anhydrous | 17.443 | 17.426 |
| (1:4 ratio with Avicel PH 102) | | |
| microcrystalline cellulose (Avicel PH 102) | 69.773 | 69.703 |
| microcrystalline cellulose (Avicel PH 105) | 8.580 | 8.571 |
| sodium starch glycolate | 4.004 | 4.000 |
| magnesium stearate (intragranular) | 0.200 | 0.200 |
| magnesium stearate (extragranular) | — | 0.200 |
| Total | 100.000 | 100.000 |

Step 1. Mixing
a. The dibasic calcium phosphate, anhydrous, microcrystalline cellulose (Avicel PH 102), microcrystalline cellulose (Avicel PH 105), and sodium starch glycolate are added to a suitable “V” blender and mixed for 15 minutes.
b. The intragranular magnesium stearate is added to the mixture from step a, and blended for 3 minutes.

Step 2. Roller Compaction
c. The blended mixture from step b is dry granulated on a suitable roller compactor, at a compression force between 8 to 12 kN/cm and at a roller speed of 3 to 6 rpm.
d. The roller-compacted material from step d is milled to a particle size suitable for tablet compression. The compression force is between 8 to 12 kN/cm at a roller speed of 3 to 6 rpm.

Step 3. Final Blending
e. The milled material is added to a suitably sized “V” blender. The remaining magnesium stearate is added to the blender and the material is mixed for 3 minutes.

C. Formulation of Amlodipine Besylate Active Blend
The following ingredients are used at the specified weight percentages to formulate an amlodipine besylate active blend composition:

| Ingredient | Wt. % |
|---|---|
| Amlodipine Besylate | 4.96 |
| Dibasic Calcium Phosphate, anhydrous | 15.79 |
| Microcrystalline Cellulose PH 102 | 70.00 |
| Microcrystalline Cellulose PH 105 | 4.96 |
| Sodium Starch Glycolate | 4.08 |
| Red or Blue Lake | 0.01 |
| Magnesium Stearate | 0.2 |
| Total | 100 |

Step 1. Blending Procedure
a. Amlodipine and an equal mass of microcrystalline cellulose (MCC) PH 105 added into a high shear mixer and mixed for 3 minutes.
b. The mixture from step a, above, is placed in a suitably sized “V” blender. MCC PH 102, sodium starch glycolate and Red or Blue Lake are added to the mixture from step a, and mixed for 15 minutes.
c. Half of the magnesium stearate is added to the mixture from step b, above, and blended for 3 minutes.

Step 2. Roller Compaction
d. The blended mixture from step c is dry granulated on a suitable roller compactor, at a compression force between 8 to 12 kN/cm and at a roller speed of 3 to 6 rpm.
e. The roller-compacted material from step d is milled to a particle size suitable for tablet compression.

Step 3. Final Active Blend

The milled material from step e is placed in a suitably sized “V” blender. The remaining magnesium stearate is added to the blender and the material is mixed for 3 minutes to obtain the final active blend.

D. Formulation of Amlodipine Besylate Plus Chlorthalidone Active Blend

The following ingredients are used at the specified weight percentages to formulate an amlodipine besylate and chlorthalidone active blend composition:

| | Percent of |
|---|---|
| Ingredient | Granulation |
| Amlodipine Besylate | 4.96 |
| Chlorthalidone | 6.67 |
| Dibasic Calcium Phosphate, anhydrous | 10.00 |
| Microcrystalline Cellulose PH 102 | 62.45 |
| Microcrystalline Cellulose PH 105 | 11.63 |
| Sodium Starch Glycolate | 4.08 |
| Red or Blue Lake | 0.01 |
| Magnesium Stearate | 0.2 |
| Total | 100 |

Step 1. Blending Procedure
a. Amlodipine and chlorthalidone and an equal mass of microcrystalline cellulose (MCC) PH 105 added into a high shear mixer and mixed for 3 minutes.
b. The mixture from step a, above, is placed in a suitably sized “V” blender. MCC PH 102, sodium starch glycolate and Red or Blue Lake are added to the mixture from step a, and mixed for 15 minutes.
c. Half of the magnesium stearate is added to the mixture from step b, above, and blended for 3 minutes.

Step 2. Roller Compaction
d. The blended mixture from step c is dry granulated on a suitable roller compactor, at a compression force between 8 to 12 kN/cm and at a roller speed of 3 to 6 rpm.
e. The roller-compacted material from step d is milled to a particle size suitable for tablet compression.

Step 3. Final Active Blend
f. The milled material from step e is placed in a suitably sized “V” blender. The remaining magnesium stearate is added to the blender and the material is mixed for 3 minutes to obtain the final active blend.

### Example 2

### Amlodipine Besylate 2.5 mg and Chlorthalidone 6 mg Tablet

Step 1. Blending Procedure
Chlorthalidone active blend and Amlodipine active blend were prepared as above, in Steps 1-3 of Examples 1 and 2, respectively.

Step 2, Tablet Compression
Tablets were manufactured using a Korsch TRP 900 multi-layer tablet press, specifically, using a 5-layer configuration.
The first layer (layer 1) contained a volume of the amlodipine final active blend to provide 2.5 mg of the amlodipine active ingredient in the final tablet.
Layers 2, 3 and 4 comprised inactive blend, as described in Example 1, above.
The last layer (layer 5) contained a volume of chlorthalidone active blend to provide 6 mg of chlorthalidone active ingredient in the final tablet.
Oval tooling was used for the compression, and the final tablets were compressed to a hardness of approximately 20 Kp.
The resulting active/inactive/active segmented tablet provides a combination drug product having 2.5 mg amlodipine and 6 mg chlorthalidone.

### Example 3

### Amlodipine Besylate 5 mg and Chlorthalidone 12 mg Tablet

Tablet Compression

Tablets were compressed using a Korsch TRP 900 multi-layer tablet press, specifically, using a 5-layer configuration. The first layer (layer 1) and last layer (layer 5) each contained a volume of the final active blend (from Step 3.f., above) to provide 2.5 mg of amlodipine and 6 mg of chlorthalidone in each layer, making a total of 5 mg of amlodipine and 12 mg chlorthalidone for the final tablet.

Layers 2, 3 and 4 contain the inactive blend, as prepared in Example 1, above.

Oval tooling was used for the compression, and the final tablets were compressed to a hardness of approximately 20 Kp.

The resulting active/inactive/active segmented tablet provides a combination drug product having 5 mg amlodipine besylate and 12 mg chlorthalidone.

It is recognized that related inventions may be within the spirit of the disclosures herein. Also no omission in the current application is intended to limit the inventors to the current claims or disclosures. While certain preferred and alternative embodiments of the invention have been set forth for purposes of disclosing the invention, modifications to the disclosed embodiments may occur to those who are skilled in the art.

1. A pharmaceutical dosage form comprising from about 1 to about 20 mg amlodipine or a pharmaceutically acceptable derivative, hydrate, isomer, metabolite, polymorph, pseudo-polymorph or salt thereof and from about 2 to about 50 mg chlorthalidone or a pharmaceutically acceptable derivative, hydrate, isomer, metabolite, polymorph, pseudo polymorph or salt thereof.

2. The dosage form as in claim 1 in which the dosage of amlodipine is from 2-15 mg.

3. The dosage form as in claim 1 in which the dosage of amlodipine is from 2.5-10 mg.

4. The pharmaceutical dosage form as in claim 1 that is a capsule.

5. The pharmaceutical dosage form as in claim 1 that is a tablet.

6. The pharmaceutical dosage form as in claim 5 that is a homogeneous tablet.

7. The pharmaceutical dosage form of claim 1, said dosage form comprising three segments: a first segment comprising a pharmacologically effective quantity of amlodipine, or a derivative, hydrate, metabolite, polymorph, or salt thereof, a second segment comprising a pharmacologically effective quantity of chlorthalidone, or a derivative, hydrate, metabolite, polymorph, or salt thereof, and a third segment comprising a composition substantially lacking pharmacological activity, said third segment being positioned between said first and second segments.

8. The pharmaceutical dosage form of claim 7 wherein said first segment further comprises chlorthalidone and said second segment further contains amlodipine, said first and second segments each containing amlodipine and chlorthalidone in a pharmacologically effective amount in similar ratios.

9. The pharmaceutical dosage form of claim 1 which comprises an inactive segment in contact with a plurality of unitary segments, said unitary segments comprising both amlodipine and chlorthalidone

10. The pharmaceutical dosage form of claim 1 comprising amlodipine and chlorthalidone as active ingredients and containing an amount of either amlodipine or chlorthalidone which has no demonstrable effect on hypertension if administered separately form the other active ingredient but which has a demonstrable additive effect to the other active ingredient when co-administered therewith.

11. The pharmaceutical dosage form as in claim 1, said dosage form further comprising an active ingredient selected from the group consisting of an anti-hypertensive agent which is not amlodipine or chlorthalidone, a potassium-retaining diuretic lacking hypotensive properties, a cholesterol lowering agent, and a vitamin.

12. A method of treating hypertension in a patient, said method comprising co-administering amlodipine and chlorthalidone.

13. The method of claim 12 wherein said co-administration comprises an administration frequency selected from the group consisting of: (a) substantially simultaneously, (b) within eight hours of each other, and (c) within 24 hours of each other.

14. A method of reducing edema induced by amlodipine, said method comprising administering chlorthalidone to a patient suffering from amlodipine-induced edema.

15. The method of treating edema caused by amlodipine as in claim 14, wherein said method further comprises treating a patient with a dose of amlodipine to produce the amlodipine-induced edema, and then relieving the amlodipine-induced edema by co-administering to the patient chlorthalidone with a second administration of amlodipine at the same dose.

16. The method of claim 15 wherein said co-administration is administering the amlodipine and chlorthalidone as part of the same dosage form.

17. The method of claim 15 wherein said co-administration of amlodipine and chlorthalidone are administered as separate dosage forms at a dosage frequency selected from the group consisting of: (a) substantially simultaneously, (b) within eight hours of each other, and (c) within 24 hours of each other.

18. The method of claim 15 in which said edema reduction is principally not from reduction of intravascular volume.

19. The method of claim 12 wherein said co-administration of amlodipine and chlorthalidone enhances the anti-hypertensive or hypotensive effect of either amlodipine or chlorthalidone.

20. The method of claim 19 wherein said effect is enhanced by providing a lower ratio of peak hypotensive effect to trough hypotensive effect as compared to a ratio of peak hypotensive effect to trough hypotensive effect ratio resulting from administering either amlodipine alone or chlorthalidone alone.

21. The method of claim 19 wherein said effect is enhanced by obtaining an improved smoothness index by co-administering amlodipine and chlorthalidone as compared to a smoothness index resulting from administering either amlodipine alone or chlorthalidone alone.

22. The method of claim 19 wherein said co-administration of amlodipine and chlorthalidone is synergistic.

23. A synergic co-formulation of amlodipine and chlorthalidone, or a co-packaging thereof.

24. The method of claim 19, said method comprising administering chlorthalidone at a dose that is insufficient to normalize blood pressure and co-administering amlodipine.

25. The method of claim 19, said method comprising administering amlodipine at a dose that is insufficient to normalize blood pressure and co-administering chlorthalidone.

26. The method of claim 19, said method comprising administering amlodipine and chlorthalidone to said patient at a dosing frequency selected from the group consisting of every second day, every third day or every fourth day during a treatment period.

27. An article of manufacture comprising at least one dosage form comprising amlodipine and chlorthalidone as active ingredients, or each active ingredient in separate dosage forms co-packaged in said article of manufacture, and further comprising a user instruction for use of the dosage form or dosage forms.

28. The article of manufacture of claim 27 wherein said instruction for use includes directing a user of the dosage form to break said dosage form in a manner to provide and administer a portion of said dosage form.

29. The method of claim 12 in which said amlodipine and chlorthalidone active ingredients are provided in separate dosage forms, said dosage forms being co-packaged.

30. The method of claim 12, said method comprising
administering a low or subtherapeutic dose of amlodipine and a subtherapeutic or low dose of chlorthalidone.

31. The method of claim 12 wherein said amlodipine and chlorthalidone are provided at doses having an effect selected from the group consisting of:
(a) the effect of said dose of amlodipine is statistically significantly higher as compared to placebo and the effect of the dose of chlorthalidone is statistically insignificant as compared to placebo but the effect of the dose of amlodipine and the effect of the dose of chlorthalidone is statistically significantly higher than compared to placebo; and
(b) the effect of said dose of chlorthalidone is statistically significantly higher than placebo and the dose of chlorthalidone is statistically insignificant compared to placebo, but the effect of the dose of amlodipine and the effect of the dose of chlorthalidone is statistically significantly higher than compared to placebo.
